(19) United States
(12) Patent Application Publication
(10) Pub. No.: US 2026/0325331 A1
(43) Pub. Date: Oct. 22, 2026

(54) MAGNETIC STENT RETRIEVAL SYSTEM AND METHODS OF USE THEREOF

(71) Applicant: Northwestern University, Evanston, IL (US)

(72) Inventors: Ahsun RIAZ, Evanston, IL (US); Riad SALEM, Evanston, IL (US); Abhinav TALWAR, Evanston, IL (US)

(21) Appl. No.: 19/113,876

(22) PCT Filed: Sep. 26, 2023
(86) PCT No.: PCT/US2023/075139

§ 371 Date: Mar. 20, 2025

Related U.S. Application Data
(60) Provisional application No. 63/377,124, filed on Sep. 26, 2022.

(51) Int. Cl. A61F 2/966; A61B 17/00; A61B 17/52; A61F 2/95; A61M 25/09

(52) U.S. Cl. CPC A61F 2/966; A61B 17/52; A61B 2017/00336; A61F 2002/9528; A61M 2025/09008

(57) ABSTRACT

Provided herein are systems comprising magnetically-couplable stents and catheters that allow for locating and retrieving the stents from within a subject. In particular, the systems herein comprise a magnetically-couplable stent and catheter, a guide wire capable of being advanced through the lumens of the stent and catheter, and a retrieval element (e.g., balloon) capable of being advanced through the lumens of the stent and catheter and stably gripping the stent. This method aims to maintain wire access across the pathology that the stent is crossing.

### CROSS-REFERENCE TO RELATED APPLICATION

The present invention claims the benefit of U.S. Provisional Patent Application Ser. No. 63/377,124, filed Sep. 22, 2022, which is hereby incorporated by reference in its entirety.

### FIELD

Provided herein are systems comprising magnetically-couplable stents and catheters that allow for locating and retrieving the stents from within a subject. In particular, the systems herein comprise a magnetically-couplable stent and catheter, a guide wire capable of being advanced through the lumens of the stent and catheter, and a retrieval element (e.g., balloon) capable of being advanced through the lumens of the stent and catheter and stably gripping the stent from the inside.

### BACKGROUND

Retrievable plastic stents are routinely placed by gastroenterology, urology, and interventional radiology (IR) clinicians to address strictures in the biliary and urinary tract. Most current methods of removing these stents include the use of snares or forceps to grab the stent. As the stent is the only device crossing the stricture, access across the stricture is lost. Additionally, obtaining access to the end of the stent to advance the snare is difficult as the stent can be opposed to the bowel lumen (or renal pelvis/urinary bladder in the case of ureteral stents). The operator then must gain access across the stricture again with an endoscope and/or wire, which has the potential of being unsuccessful.

Plastic stent patency requires that stents be exchanged frequently. Limitations on the current retrieval technologies can result in failure to locate and/or successfully retrieve the stent, prolonged stent retrieval times, and loss of access across the stricture.

### SUMMARY

Provided herein are systems comprising magnetically-couplable stents and catheters that allow for locating and retrieving the stents from within a subject. In particular, the systems herein comprise a magnetically-couplable stent and catheter, a guide wire capable of being advanced through the lumens of the stent and catheter, and a retrieval element (e.g., balloon) capable of being advanced through the lumens of the stent and catheter and stably gripping the stent from the inside.

In some embodiments, provided herein are stent removal catheters comprising a magnetic-alignment element capable of being magnetically coupled to a portion of a stent. In some embodiments, the stent removal catheter comprises: (a) an outer sheath, wherein the outer sheath comprises an outer sheath lumen, a distal end for inserting into a subject, and a proximal end which remains outside the subject and can be manipulated by a clinician; and (b) a stent engagement catheter, wherein the stent engagement catheter is configured to reside within the outer sheath lumen, and wherein the stent engagement catheter comprises an inner lumen and a distal end; wherein the magnetic-alignment element is located on the distal end of the stent engagement catheter; and wherein the distal end of the stent engagement catheter is configured to be extended from the distal end of the outer sheath. In some embodiments, the magnetic-alignment element comprises a permanent magnet. In some embodiments, the magnetic-alignment element comprises a ferromagnetic material. In some embodiments, the stent engagement catheter is 2-6 French in diameter.

In some embodiments, provided herein are magnetically-removable stents comprising a magnetic-alignment element. In some embodiments, the magnetically-removable stent comprises a proximal end, wherein the proximal end provides a leading edge for the removal of the magnetically-removable stent from a subject, and wherein the magnetic-alignment element is located on the proximal end of the magnetically-removable stent. In some embodiments, the magnetic-alignment element comprises a permanent magnet. In some embodiments, the magnetic-alignment element comprises a ferromagnetic material.

In some embodiments, provided herein are systems comprising: (a) a magnetically-removable stent comprising a stent lumen and a first magnetic-alignment element; (b) a stent engagement catheter comprising an inner catheter lumen and a second magnetic-alignment element capable of aligning with and being magnetically coupled to the first magnetic-alignment element; (c) an outer sheath comprising an outer sheath lumen, the stent engagement catheter configured to reside within the other sheath lumen and capable of extending from a distal end of the outer sheath; (d) a guidewire capable of extending through the stent lumen and the inner catheter lumen; and (e) a retrieval element, capable of being deployed along the guidewire through the outer sheath lumen and into the stent lumen, and capable of securely engaging with the stent. In some embodiments, the stent engagement catheter comprises a distal end configured for extending from the distal end of the outer sheath, and wherein the second magnetic-alignment element is located on the distal end of the stent engagement catheter. In some embodiments, the magnetically-removable stent comprises a proximal end, wherein the proximal end provides a leading edge for the removal of the magnetically-removable stent from a subject, and wherein the first magnetic-alignment element is located on the proximal end of the magnetically-removable stent. In some embodiments, the first magnetic-alignment element comprises a permanent magnet and the second magnetic-alignment element comprises a ferromagnetic material. In some embodiments, the first magnetic-alignment element comprises a ferromagnetic material and the second magnetic-alignment element comprises a permanent magnet. In some embodiments, the distal end of the stent engagement catheter is configured to reside within the stent lumen at the proximal end of the magnetically-removable stent, thereby aligning the first and second magnetic-alignment elements for magnetic coupling. In some embodiments, the retrieval element is a balloon. In some embodiments, the balloon is capable of adopting a deflated conformation in which the balloon can be deployed along the guidewire through the outer sheath lumen and into the stent lumen; and wherein the balloon is capable of adopting an inflated conformation in which the balloon presses against the interior of the stent lumen, thereby securely engaging with the stent. In some embodiments, when the balloon is retrieved along the guidewire when in an inflated conformation within the stent lumen, the secure engagement of the balloon with the stent results in the stent being retrieved through the outer sheath lumen.

In some embodiments, provided herein are methods of removing a magnetically-engageable stent from a treatment site in a lumen of a subject, wherein the stent comprises a stent lumen and a first magnetic engagement element located on a proximal end of the stent, the method comprising: (a) inserting a stent removal catheter into the lumen of the subject and placing a distal end of the stent removal catheter adjacent to a proximal end of the magnetically-engageable stent, wherein the stent removal catheter comprises: (i) a stent engagement catheter comprising an inner catheter lumen and a second magnetic-alignment element capable of aligning with and being magnetically coupled to the first magnetic-alignment element; and (ii) an outer sheath comprising an outer sheath lumen, the stent engagement catheter configured to reside within the other sheath lumen and capable of extending from a distal end of the outer sheath; (b) extending the stent engagement catheter from the distal end of the outer sheath and contacting the first magnetic engagement element with the second magnetic engagement element, thereby stably engaging the stent with the stent engagement catheter; (c) extending a guidewire through the inner catheter lumen and into the stent lumen; (d) disengaging the stent engagement catheter from the stent and withdrawing the stent engagement catheter through the outer sheath lumen; (e) deploying a retrieval element along the guidewire through the outer sheath lumen and into the stent lumen; (f) converting the retrieval element into an expanded conformation, in which the retrieval element stably engages the inner wall of the stent lumen; and (g) withdrawing the retrieval element into the outer sheath lumen, thereby retrieving the stent into the outer sheath lumen. In some embodiments, the guidewire remains extended across the treatment site following retrieval of the stent.

### BRIEF DESCRIPTION OF THE DRAWINGS

FIG. 1a. A drawing depicting the distal end of an exemplary catheter (1) having a magnetic-alignment element (2) mounted at the distal terminus of the catheter.

FIG. 1b. A drawing depicting an exemplary stent (4) with a magnetic-alignment element (3) mounted adjacent to the opening of the stent lumen.

FIG. 1c. A drawing depicting a pigtail stent (10) with a magnetic-alignment element (13) mounted adjacent to the opening of the stent lumen at the leading edge of the stent for retrieval.

FIG. 1d. A drawing of an exemplary catheter (1) and stent (4) with complementary magnetic-alignment elements (cylindrical magnet on the catheter (2) and ferromagnetic element on the stent (3)), immediately prior to alignment and magnetic engagement.

FIG. 1e. A drawing of an exemplary catheter (1) and stent (2) aligned for stent retrieval and with complementary magnetic-alignment elements (2/3) engaged.

FIG. 1f. A drawing of magnetically-engaged catheter and stent with guidewire (5) extending through the catheter lumen and into the stent lumen.

FIG. 1g. A drawing depicting an exemplary catheter that has been disengaged from the exemplary stent (4), following insertion of the guidewire (5) through the catheter and stent lumens.

FIG. 1h. A drawing depicting a non-compliant balloon (7) that has been advanced through the catheter lumen along guidewire, into the stent lumen, and inflated within the stent lumen, thereby stably engaging the stent from within.

FIG. 2a. A drawing of an exemplary angled catheter (1) and stent (4) with complementary magnetic-alignment elements (2 is the cylindrical magnet on the catheter and 3 is the ferromagnetic element on the stent), immediately prior to alignment and magnetic engagement.

FIG. 2b. A drawing of an exemplary catheter (1) and stent (2) aligned for stent retrieval and with complementary magnetic-alignment elements (2 and 3) engaged. The guidewire (5) is extending through the catheter lumen and into the stent lumen.

FIG. 2c. A drawing depicting an exemplary catheter that has been disengaged from the exemplary stent (4), following insertion of the guidewire (5) through the catheter and stent lumens.

FIG. 2d. A drawing depicting a non-compliant balloon (7) that has been advanced through the catheter lumen along guidewire, into the stent lumen, and inflated within the stent lumen, thereby stably engaging the stent from within. The sheath (6) which will be used to serve as a conduit for retrieving the stent is also seen.

FIG. 3. A drawing depicting a pigtail stent (10) with its end-hole (11) and side-holes (12). There is a magnetic-alignment element (13) mounted on an enlarged side hole (14) at the straight segment of the leading edge of the stent for retrieval.

### DETAILED DESCRIPTION

Provided herein are systems comprising magnetically-couplable stents and catheters that allow for locating and retrieving the stents from within a subject. In particular, the systems herein comprise a magnetically-couplable stent and catheter, a guide wire capable of being advanced through the lumens of the stent and catheter, and a retrieval element (e.g., balloon) capable of being advanced through the lumens of the stent and catheter and stably gripping the stent.

In some embodiments, provided herein are stents for use in the biliary or urinary system. In some embodiments, the stents herein find use, for example, in relieving pathologic strictures or obstructions in a lumen (e.g., ureter, bile ducts, pancreatic ducts, etc.) of a subject. In some embodiments, a stent is a ureteral stent and finds use in treating or preventing a ureteral stricture or obstructions, allowing urine to properly flow from the kidneys into the bladder. In some embodiments, a stent is a biliary stent and finds use in treating or preventing occlusions, strictures, and/or leaks in a bile duct. In some embodiments, a stent is a pancreatic stent and finds use in treating or preventing narrowed, blocked, or leaking pancreatic ducts. Embodiments herein are not limited by the type of stent, the locations of placement, or the indication that the stenting is intended to treat/prevent.

In some embodiments, the body of the stent comprises a thin and flexible material that allows the stent to approximate the inner wall of a lumen it is placed within. In some embodiments, the body of the stent is sufficiently resistant to compression to allow it to prevent and/or correct a stricture in the lumen it is placed within.

In some embodiments, the stent body comprises a flexible, non-magnetic material, such as silicone, plastic (e.g., polyurethane, polyethylene, or blends thereof), Teflon, or other polymers/elastomers.

In some embodiments, the stent body comprises a tubular (cylindrical) shape with a length of 1-50 cm (e.g., 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 25 cm, 30 cm, 35 cm, 40 cm, 55 cm, 50 cm, or ranges therebetween) and a diameter of 2-15 Fr (e.g., 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, or ranges therebetween). In some embodiments, the length and diameter of the stent is selected based on the subject and location of placement. Shapes and features of stents, particular to their particular placement locations will be understood by those in the field. In some embodiments, stents can be reenforced (e.g., with metal) for increased radial force as they do not have to be folded on themselves for retrieval.

A feature of embodiments herein is the inclusion of a magnetic alignment element within the stent. The magnetic alignment element is a piece of ferromagnetic material, or a permanent magnet located on the stent. In some embodiments, the magnetic alignment element of the stent finds use in a pair with a complementary magnetic alignment element on a stent engagement catheter. In particular embodiments, the first of the two magnetic alignment elements is ferromagnetic and a second is a permanent magnet. In some embodiments, the permanent magnet is on the stent and the ferromagnetic element is on the retrieval catheter. In some embodiments, the permanent magnet is on the retrieval catheter and the ferromagnetic element is on the stent. In some embodiments, both of the magnetic alignment elements are permanent magnets which are oriented to engage when properly positioned with respect to one another.

The magnetic alignment element is positioned on the stent in a location that is accessible to a catheter designed for stent engagement. In some embodiments, the magnetic alignment element is positioned at or near the proximal end of the stent (e.g., the end nearest to access the stent for removal). In some embodiments, the magnetic alignment element is positioned at or near the leading removal edge of the stent. In some embodiments, the magnetic alignment element approximates or forms all or a portion of the proximal end of the stent.

In some embodiments, the magnetic attraction between the complementary magnetic alignment elements is sufficiently strong to cause proper orientation and alignment of the retrieval catheter with the stent, upon bringing the distal end of the catheter into sufficient proximity (e.g., <3 cm, <2 cm, <1 cm, <0.5 cm, etc.) with the proximal end of the stent. In some embodiments, the magnetic attraction between the complementary magnetic alignment elements is sufficiently weak to allow disengagement of the complementary magnetic alignment elements (and the catheter from the stent) without significant disruption of the position of the stent within the treatment location (e.g., moving the stent positions within the treatment location less than 1 cm (e.g., <1 cm, <9 mm, <8 mm, <7 mm, <6 mm,5 mm, <4 mm, <3 mm, <2 mm, <1 mm).

In some embodiments, provided herein are catheters for the retrieval of stents from treatment sites in the biliary, urinary, or pancreatic system (i.e., stent retrieval catheters). In some embodiments, the retrieval catheters comprise an outer sheath and a stent engagement catheter. In some embodiments, both the outer sheath and the stent engagement catheter are elongated tubes (cylinders) with inner lumens. In some embodiments, the stent engagement catheter is shaped and sized to reside within the lumen of the outer sheath. In some embodiments, the stent engagement catheter can be deployed through the outer sheath. In some embodiments, the stent engagement catheter can be inserted through an opening at the proximal end of the outer sheath. In some embodiments, the distal end of the stent engagement catheter is configured to be deployed (extended from) the distal end of the outer sheath. In some embodiments, the stent engagement catheter and outer sheath are separable devices that are collectively referred to herein as the retrieval catheter. In some embodiments, a catheter is of any suitable shape and size. A catheter may be straight, angled (e.g., 10°, 20°, 30°, 40°, 50°, or ranges therebetween), curved, or any suitable shape.

In some embodiments, the outer sheath comprises an outer diameter suitable to traverse a lumen or lumens of a subject from the catheter insertion site to the location of the stent (i.e., treatment site). The outer diameter of the outer sheath may be from about 3-16 Fr (e.g., 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, or ranges therebetween). In some embodiments, the inner diameter of the outer sheath is suitable to accommodate the outer diameter of the stent engagement catheter, the other diameter of the stent, a guidewire and retrieval element (not necessarily simultaneously). In some embodiments, the inner diameter of the outer sheath is 1 mm to 5 mm greater than the outer diameter of the stent.

In some embodiments, the outer sheath comprises a distal end for insertion through a lumen of a subject (e.g., bile duct, pancreatic duct, ureter, etc.) and a proximal end for manipulation/control by an operator (e.g., clinician). In some embodiments, the proximal end of the outer sheath is maneuvered through lumen(s) within a subject to place the distal end of the outer sheath in proximity (e.g., <5 cm, <4 cm, <3 cm, <2 cm, <1 cm) with the proximal end of a stent at a treatment site. In some embodiments, the outer sheath is used to deliver and/or retrieve various components (e.g., stent engagement catheter, guidewire, retrieval element, etc.) of the retrieval systems herein to/from the treatment site.

In some embodiments, the stent engagement catheter comprises an outer diameter suitable reside within the outer sheath and to be deployed from the distal end of the sheath and/or retracted through the outer sheath. In some embodiments, the outer diameter of the stent engagement catheter may be from about 2-15 Fr (e.g., 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, or ranges therebetween). In some embodiments, the stent engagement catheter comprises a tubular (cylindrical) shape with an internal lumen. In some embodiments, the lumen of the stent engagement catheter is of sufficient diameter to allow a guidewire to be deployed and retrieved through the stent engagement catheter lumen.

A feature of embodiments herein is the inclusion of a magnetic alignment element at the distal end of the stent engagement catheter. The magnetic alignment element is a piece of ferromagnetic material or a permanent magnet located on the stent engagement catheter. In some embodiments, the magnetic alignment element of the stent engagement catheter finds use in a pair with a complementary magnetic alignment element on the stent. In particular embodiments, a first of the two magnetic alignment elements is ferromagnetic and a second is a permanent magnet. In some embodiments, the permanent magnet is on the stent and the ferromagnetic element is on the stent engagement catheter. In some embodiments, the permanent magnet is on the stent engagement catheter and the ferromagnetic element is on the stent. In some embodiments, both magnetic alignment elements are permanent magnets which are oriented to engage when properly positioned with respect to one another.

The magnetic alignment element is positioned on the stent engagement catheter in a location that can access the stent when the stent engagement catheter is deployed from the distal end of the outer sheath. In some embodiments, the magnetic alignment element is positioned at or near the distal end of the stent engagement catheter. In some embodiments, the magnetic alignment element approximates or forms all or a portion of the distal end of the stent engagement catheter.

In some embodiments, stent retrieval systems herein comprise a guidewire. The guidewire is capable of being deployed through the inner lumen of the stent engagement catheter (e.g., when the stent engagement catheter is extended from the distal end of the outer sheath. In some embodiments, the guidewire is capable of being deployed into and through a stent (e.g., when the stent and the stent engagement catheter are magnetically coupled. In some embodiments, after deployment of the guidewire into the stent, the stent engagement catheter can be withdrawn into the outer sheath, leaving the guidewire in place within the stent (e.g., extending through and beyond the stent or terminating within the stent). In some embodiments, the stent engagement catheter can be completely withdrawn through the outer sheath, removing the stent engagement catheter from a subject, while leaving the guidewire in place within the outer sheath and stent.

In some embodiments, the guidewire is composed of a non-ferromagnetic material (e.g., plastic, a polymer, an elastomer, etc.). In some embodiments, the guidewire is composed of a flexible material, capable of being snaked through the lumens of the devices herein while deployed in a subject.

In some embodiments, the guidewire is 3 mm or less in diameter (e.g., 3 mm, 2 mm, 1 mm, 0.5 mm, or less).

In some embodiments, the guidewire provides a structure for deployment of the retrieval element along. In some embodiments, the retrieval element is advanced along the guidewire, through the lumen of the outer sheath, and into the stent.

In some embodiments, the guidewire is separate component from the outer sheath, stent engagement catheter, stent, and retrieval elements. In some embodiments, the guidewire can be left in place within a lumen of a subject when the other elements are removed. In some embodiments, access to the treatment site (e.g., across a potential occlusion and/or stricture) is maintained by the guidewire when the stent and/or other components described herein are removed.

Embodiments herein comprise a retrieval element for use in removing a stent from within a treatment site in a subject. In some embodiments, the retrieval element is a component of the system herein that can be deployed into a stent in a first conformation (e.g., collapsed, deflated, low profile, etc.). When in the first conformation and being deployed into the stent, the retrieval element does not disturb the orientation or location of the stent. In some embodiments, when in the first conformation, the retrieval element does not contact the stent when being deployed into the stent. In some embodiments, a retrieval element is capable of adopting (e.g., being manipulated into) a second conformation (e.g., expanded, inflated, high profile, etc.). When in the second conformation and deployed within the lumen of the stent, the retrieval element is in contact with the inner wall of the stent. In some embodiments, when in the second conformation, the retrieval element is stably engaged with the stent. In some embodiments, expansion of the retrieval element within the stent from the first conformation (e.g., collapsed, deflated, low profile, etc.) into the second conformation (e.g., expanded, inflated, high profile, etc.) causes the retrieval element to become stably engaged with the stent (e.g., wedged within the stent). In some embodiments, pressure exerted onto the stent by the expansion of the retrieval element within the stent causes the retrieval element to stably engage the stent.

In some embodiments, once the retrieval element has stably engaged the stent (e.g., while remaining in the second conformation (e.g., expanded, inflated, high profile, etc.), if the retrieval element is withdrawn back into the outer sheath, the stent is withdrawn by the retrieval element into the outer sheath.

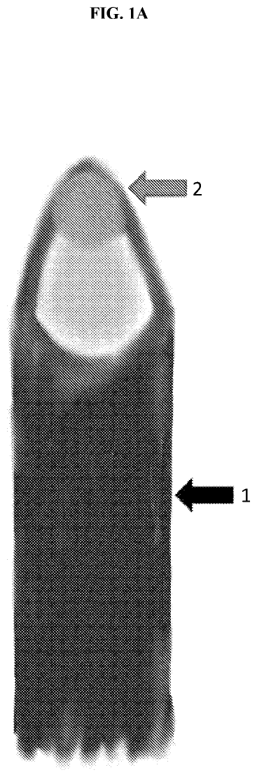

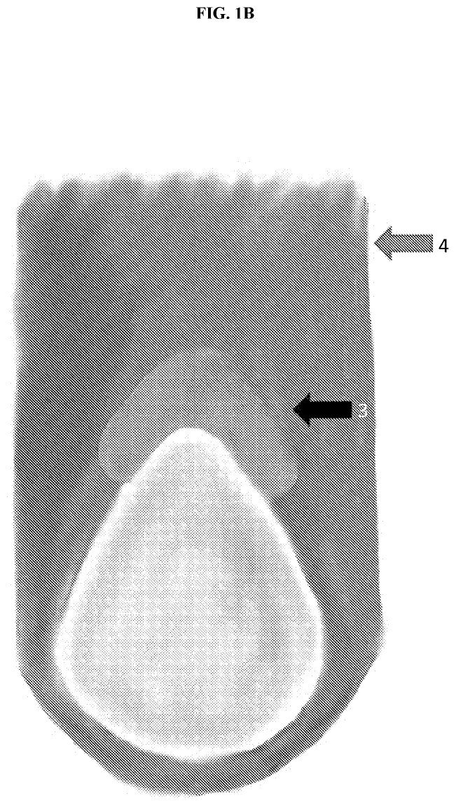

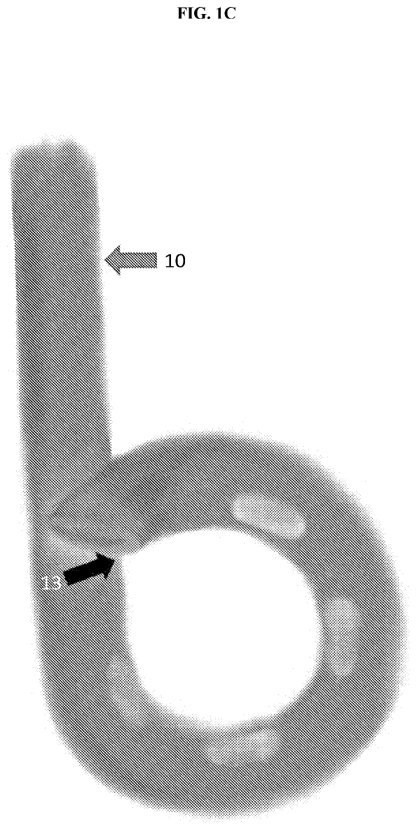

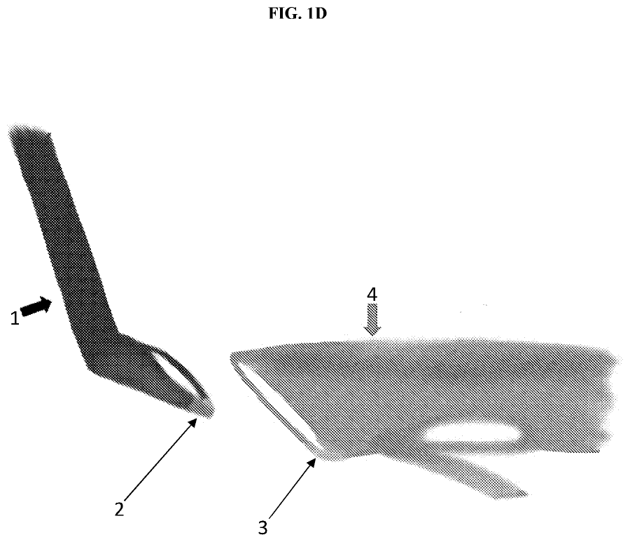

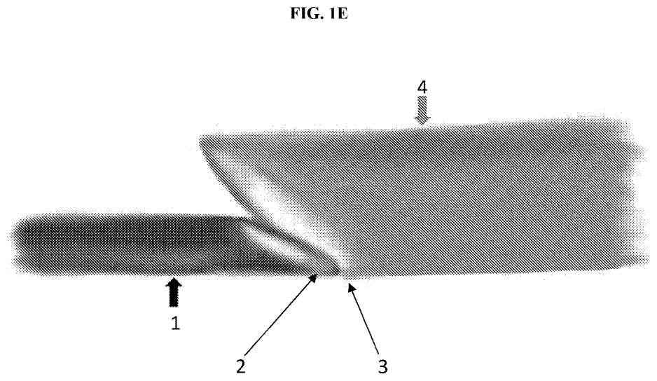

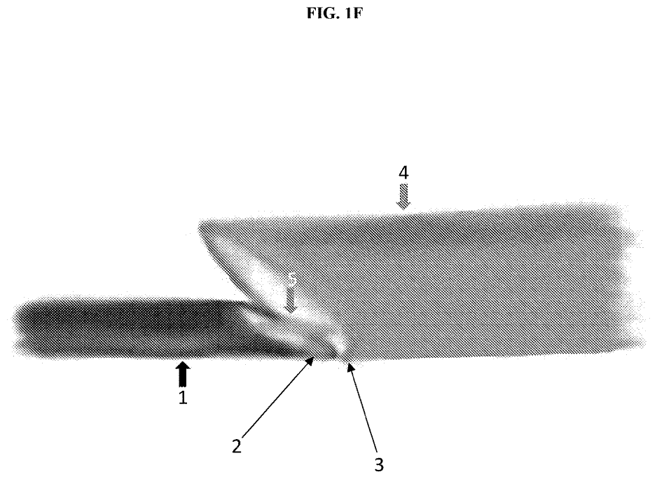

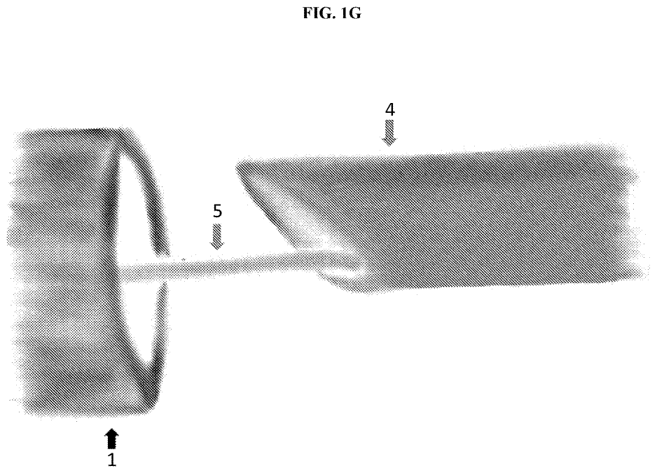

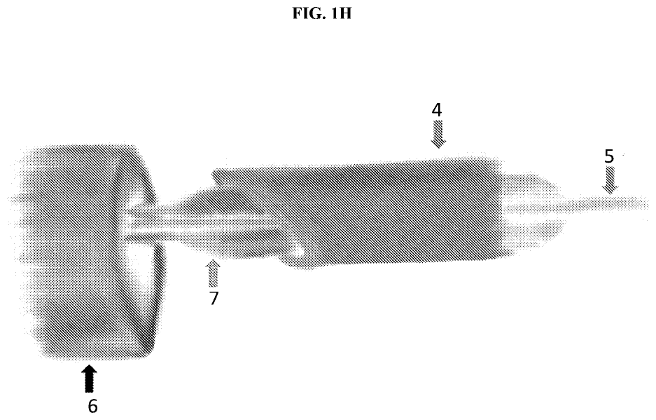

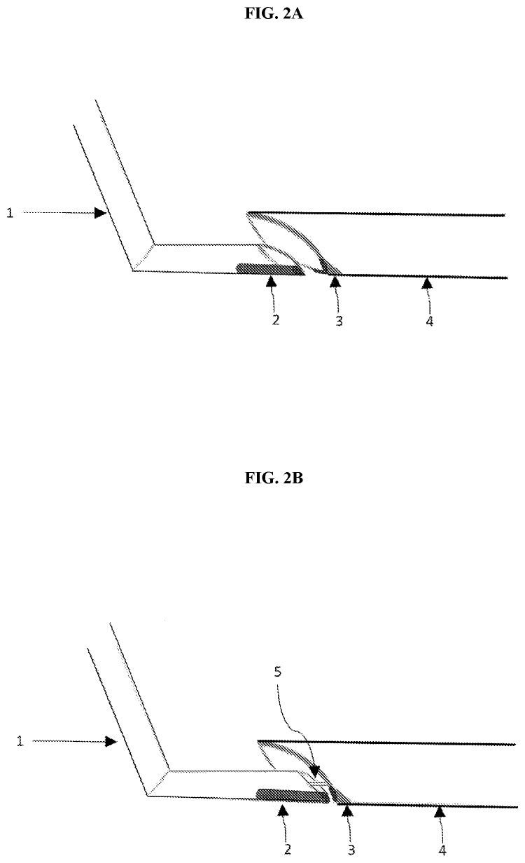

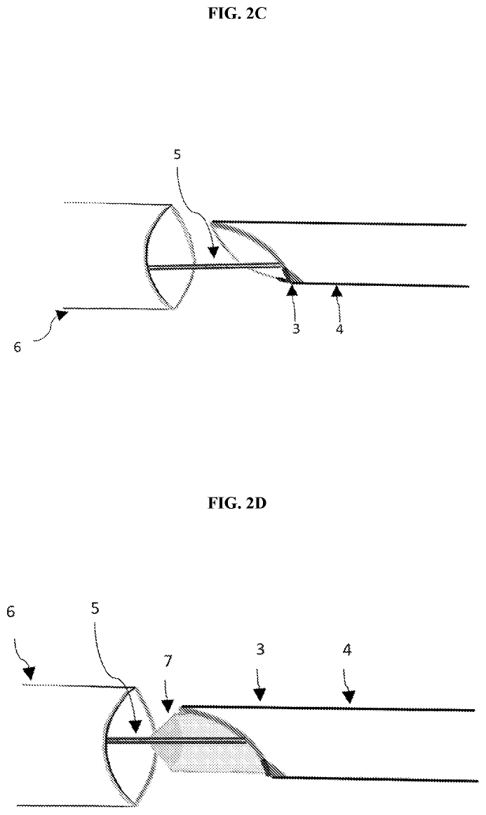

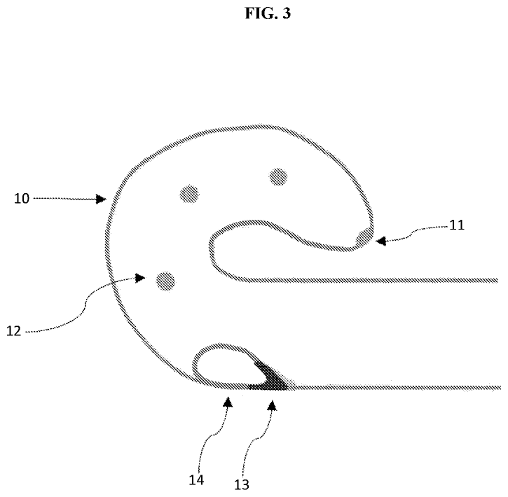

In some embodiments, the retrieval element (e.g., balloon) is advanced over the guidewire into the stent. As seen in FIG. 1G, care is taken so the tapered edge of the balloon protrudes from the end of the stent (to ease retraction into the sheath). In some embodiments, the balloon is then inflated using commercially available balloon inflaters (e.g., Encore 26 Balloon Inflator [Boston Scientific; Marlborough; MA]). The balloon is then pin-pulled over the wire. As the balloon has gripped the stent from the inside, the stent is retracted with the balloon into the sheath and out of the patient. The wire remains in position across the stricture. Once the balloon/stent are removed, the balloon can be deflated outside the patient and the stent separated from the balloon catheter. The balloon can then be reused in the same case if needed.

The retrieval element can be any components capable of adopting both first and second confirmations, such that it can be deployed without perturbing the stent and retrieved when stably engaging the stent. In particular embodiments, the retrieval element is a balloon. In some embodiments, the balloon is non-compliant. In some embodiments, the balloon comprises a material, such as a polymer, selected from, for example, Nylon, PEEK, Pebax, or the like. In some embodiments, the balloon is capable of inflation to a diameter greater than the inner diameter of the stent. In some embodiments, upon full inflation of the balloon, pressure is exerted on the interior of the stent, thereby locking the balloon in place within the stent. When the inflated balloon is withdrawn back into the outer sheath, the stent, which is securely engaged by the balloon due to the pressure is exerted on the interior of the stent by the balloon, is withdrawn therewith.

In some embodiments, a stent is placed to prevent, relieve, correct, or otherwise treat a stricture occurring in a lumen (e.g., ureter, bile duct, etc.) of a subject. In some embodiments, a stent is placed to treat or prevent obstructions due to, for example, blood clots, inflammatory bowel disease (IBD), scar tissue build up (e.g., caused by endometriosis or other conditions), tumors, ureteral stones, ureteropelvic junction obstruction (e.g., caused by narrow ureters).

In some embodiments, the catheter(s) herein are advanced into the subject (and through the lumens of a subject) through the working channel of the endoscope. In some embodiments, the catheter and the stent are aligned using endoscopy and/or fluoroscopy.

In some embodiments, the stent retrieval methods described herein are performed by an human operator. In some embodiments, one or more (e.g., all) of the steps of a method described herein are automated (e.g., performed by a robotic instrument or system).

In some embodiments, the devices and methods herein find use in endobiliary ablation. Stretching the bile duct open after ablation is a key to clinical success. Endobiliary ablation uses thermal energy to heat the stricture, ablating the fibrosis (scar) or tumor (cancer). The plastic stents placed in tandem allow prolonged dilatation of the stricture following the ablation and the stents serve as a scaffold for the new scarring to form around. In the absence of stents, this scarring can be severe and cause worsening of the stricture.

1. A stent removal catheter comprising a magnetic-alignment element capable of being magnetically coupled to a portion of a stent.

2. The stent removal catheter of claim 1, wherein the stent removal catheter comprises:
(a) an outer sheath, wherein the outer sheath comprises an outer sheath lumen, a distal end for inserting into a subject, and a proximal end which remains outside the subject and can be manipulated by a clinician; and
(b) a stent engagement catheter, wherein the stent engagement catheter is configured to reside within the outer sheath lumen, and wherein the stent engagement catheter comprises an inner lumen and a distal end;
wherein the magnetic-alignment element is located on the distal end of the stent engagement catheter; and
wherein the distal end of the stent engagement catheter is configured to be extended from the distal end of the outer sheath.

3. The stent removal catheter of claim 1, wherein magnetic-alignment element comprises a permanent magnet.

4. The stent removal catheter of claim 1, wherein magnetic-alignment element comprises a ferromagnetic material.

5. The stent removal catheter of claim 1, wherein the stent engagement catheter is 2-6 French in diameter.

6. A magnetically-removable stent comprising a magnetic-alignment element.

7. The magnetically-removable stent of claim 6, wherein the stent magnetically-removable stent comprises a proximal end, wherein the proximal end provides a leading edge for the removal of the magnetically-removable stent from a subject, and wherein the magnetic-alignment element is located on the proximal end of the magnetically-removable stent.

8. The magnetically-removable stent of claim 6, wherein magnetic-alignment element comprises a permanent magnet.

9. The magnetically-removable stent of claim 6, wherein magnetic-alignment element comprises a ferromagnetic material.

10. A system comprising:
(a) a magnetically-removable stent comprising a stent lumen and a first magnetic-alignment element;
(b) a stent engagement catheter comprising an inner catheter lumen and a second magnetic-alignment element capable of aligning with and being magnetically coupled to the first magnetic-alignment element;
(c) an outer sheath comprising an outer sheath lumen, the stent engagement catheter configured to reside within the other sheath lumen and capable of extending from a distal end of the outer sheath;
(d) a guidewire capable of extending through the stent lumen and the inner catheter lumen; and
(e) a retrieval element, capable of being deployed along the guidewire through the outer sheath lumen and into the stent lumen, and capable of securely engaging with the stent.

11. The system of claim 10, wherein the stent engagement catheter comprises a distal end configured for extending from the distal end of the outer sheath, and wherein the second magnetic-alignment element is located on the distal end of the stent engagement catheter.

12. The system of claim 11, wherein the magnetically-removable stent comprises a proximal end, wherein the proximal end provides a leading edge for the removal of the magnetically-removable stent from a subject, and wherein the first magnetic-alignment element is located on the proximal end of the magnetically-removable stent.

13. The system of claim 12, wherein the first magnetic-alignment element comprises a permanent magnet and the second magnetic-alignment element comprises a ferromagnetic material.

14. The system of claim 12, wherein the first magnetic-alignment element comprises a ferromagnetic material and the second magnetic-alignment element comprises a permanent magnet.

15. The system of claim 10, wherein the distal end of the stent engagement catheter is configured to reside within the stent lumen at the proximal end of the magnetically-removable stent, thereby aligning the first and second magnetic-alignment elements for magnetic coupling.

16. The system of claim 10, wherein the retrieval element is a balloon.

17. The system of claim 16, wherein the balloon is capable of adopting a deflated conformation in which the balloon can be deployed along the guidewire through the outer sheath lumen and into the stent lumen; and wherein the balloon is capable of adopting an inflated conformation in which the balloon presses against the interior of the stent lumen, thereby securely engaging with the stent.

18. The system of claim 17, wherein when the balloon is retrieved along the guidewire when in an inflated conformation within the stent lumen, the secure engagement of the balloon with the stent results in the stent being retrieved through the outer sheath lumen.

19. A method of removing a magnetically-engageable stent from a treatment site in a lumen of a subject, wherein the stent comprises a stent lumen and a first magnetic engagement element located on a proximal end of the stent, the method comprising:
(a) inserting a stent removal catheter into the lumen of the subject and placing a distal end of the stent removal catheter adjacent to a proximal end of the magnetically-engageable stent, wherein the stent removal catheter comprises:
(i) a stent engagement catheter comprising an inner catheter lumen and a second magnetic-alignment element capable of aligning with and being magnetically coupled to the first magnetic-alignment element; and
(ii) an outer sheath comprising an outer sheath lumen, the stent engagement catheter configured to reside within the other sheath lumen and capable of extending from a distal end of the outer sheath;
(b) extending the stent engagement catheter from the distal end of the outer sheath and contacting the first magnetic engagement element with the second magnetic engagement element, thereby stably engaging the stent with the stent engagement catheter;
(c) extending a guidewire through the inner catheter lumen and into the stent lumen;
(d) disengaging the stent engagement catheter from the stent and withdrawing the stent engagement catheter through the outer sheath lumen;
(e) deploying a retrieval element along the guidewire through the outer sheath lumen and into the stent lumen;
(f) converting the retrieval element into an expanded conformation, in which the retrieval element stably engages the inner wall of the stent lumen; and
(g) withdrawing the retrieval element into the outer sheath lumen, thereby retrieving the stent into the outer sheath lumen.

20. The method of claim 19, wherein the guidewire remains extended across the treatment site following retrieval of the stent.
